(19) United States
(12) Patent Application Publication
(10) Pub. No.: US 2026/0035719 A1
(43) Pub. Date: Feb. 5, 2026

(54) SYNTHESIS METHOD FOR ITACONATE DRIVEN BY THIOESTERASE

(71) Applicant: National Tsing Hua University, Hsinchu (TW)

(72) Inventors: Roa Pu Claire Shen, Hsinchu (TW); Ryan S. Wang, Hsinchu (TW); Siang-Wun Siao, Hsinchu (TW); Jessica C. Wang, Hsinchu (TW); Patrick Y. Lin, Hsinchu (TW); Yu-Wei Liao, Hsinchu (TW)

(21) Appl. No.: 19/285,241

(22) Filed: Jul. 30, 2025

Related U.S. Application Data
(60) Provisional application No. 63/678,639, filed on Aug. 2, 2024.

(51) Int. Cl. C12P 7/44; C12N 9/10; C12N 9/24; C12N 9/88; C12N 15/52; C12N 15/70

(52) U.S. Cl. CPC C12P 7/44; C12N 9/13; C12N 9/2402; C12N 9/88; C12N 15/52; C12N 15/70; C12N 2800/101

(57) ABSTRACT

The present disclosure provides a synthesis method for itaconate driven by thioesterase. The present disclosure provides a novel method for synthesizing itanonate driven by thioester hydrolysis through experiments illustrated in the examples.

### CROSS-REFERENCE TO RELATED APPLICATION

This application claims priority of Provisional application No. 63/678,639, filed on Aug. 2, 2024, the content of which is incorporated herein in its entirety by reference.

### BACKGROUND OF THE INVENTION

### 1. Field of the Invention

The present invention relates to a synthesis method for itaconate driven by thioesterase.

### 2. The Prior Art

Recently, recycled materials obtained by converting biomass have received more and more attention. Itaconate (IA) is a C5 dicarboxylic acid with bifunctional groups and an unsaturated double bond, making it useful in the field of polymer synthesis. Itaconate has once been proposed by the US Department of Energy (DOE) to be the top 12 value added bio-based chemicals. Moreover, IA plays a significant role in sustainability for being a renewable substitute of a variety of petroleum-based chemical such as acrylic acid and methacrylic acid, so as to reduce the reliance on oil-based resources and long-term environment damages. IA has the following chemical structure:

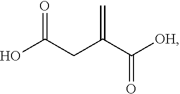

and is an essential precursor for many products (e.g., polypropylene fibers, rubber, artificial diamonds, and lenses), which are widely used in the chemical industry. Traditionally, IA has been isolated from Aspergillus terreus. However, the growth of Aspergillus terreus is slow and Aspergillus terreus cannot produce IA during the sporulation period. Therefore, there is a need in the industry for a novel method for production of IA.

Itaconate is a commercially valuable platform chemical with wide applications in the synthetic polymer industry. Production of itaconate has long relied on the decarboxylation of TCA cycle intermediate cis-aconitate, forming the methylene-attached dicarboxylic acid with one carbon lost in the process. Despite its success in the native producers, the complex life cycle of fungi and the desire for additional traits such as photosynthetic biosynthesis have led to numerous efforts in engineering the fungal pathway in recombinant hosts. The insolubility of fungal cis-aconitate decarboxylase and necessity of TCA cycle disruption often post challenges to the recombinant strain development and operation cost. The inevitable loss of cis-aconitate to the reversible interconversion of citrate and isocitrate also adds to the difficulty of securing precursor supply, which was recently tackled by replacing the native aconitase with the engineered dehydratase. Itaconate detoxification pathway had evolved in certain infectious bacteria such as Yersinia pestis, Pseudomonas aeruginosa, and Burkholderia xenovorans to combat the growth suppression caused by the itaconate inhibition of the glyoxylate shunt and the methylcitrate cycle. During infection, the natural defense mechanism of mammalian macrophage works through the synthesis and release of itaconate, which allosterically inactivates isocitrate and methylcitrate lyase and disables the pathogen's ability to assimilate acetyl-CoA or propionyl-CoA in the glucose limiting condition. Degradation of itaconate initiates with CoA activation of the dicarboxylic acid via itaconyl-CoA transferase (Ict). The resulting itaconyl-CoA is then converted into (S)-citramalyl-CoA via itaconyl-CoA hydratase (Ich) possessing both the itaconyl-CoA isomerase and mesaconyl-CoA hydratase activity module. Lastly, cleavage of(S)-citramalyl-CoA into acetyl-CoA and pyruvate occurs through the action of(S)-citramalyl-CoA lyase (Ccl), which has homologues commonly found in the 3-hydroxypropionate cycle.

Escherichia coli (usually abbreviated: E. coli) is a bacterium in the gut of humans and animals, mainly parasitic in the large intestine, accounting for about 0.1% of intestinal bacteria. Escherichia coli is a Gram-negative, facultative anaerobic, rod-shaped, coliform bacterium of the genus Escherichia.

Although researchers in the field have found that E. coli can be used to produce IA. However, in the E. coli strains, in order to accumulate the precursors for producing IA, it is necessary to inhibit the important gene icd which encodes isocitrate dehydrogenase in the citric acid cycle, and inhibiting the icd gene causes α-ketoglutaric acid (α-KG) and glutamate auxotrophs. However, such interference often results in glutamate deficiency, making the strains unable to grow and produce IA in basal medium.

In view of this long-standing problem, the conventional technique is to provide the essential amino acid (i.e., glutamate) which cannot be synthesized by the IA producing strain inhibiting the icd gene by adding a large amount of yeast extract or glutamate to maintain growth and help the strain grow.

In order to provide a different itaconate production pathway from the traditional one to hopefully overcome the above-mentioned problems, those skilled in the art urgently need to develop a novel method for synthesizing itanonate for the benefit of a large group of people in need thereof.

### SUMMARY OF THE INVENTION

In order to solve the foregoing problems, an objective of the present invention is to provide a method for synthesizing itaconate driven by thioesterase, comprising initiating pyruvate and acetyl-CoA condensation to form (S)-citramalyl-CoA through a reversed degradation pathway, followed by dehydration and isomerization of the (S)-citramalyl-CoA into itaconyl-CoA, and then hydrolysis of the itaconyl-CoA into itaconate driven by thioesterase.

According to an embodiment of the present invention, the thioesterase is from Escherichia coli.

According to an embodiment of the present invention, the Escherichia coli is Escherichia coli strain JCL299 (ΔldhA ΔadhE ΔfrdBC Δpta).

According to an embodiment of the present invention, the Escherichia coli strain JCL299 (ΔldhA ΔadhE ΔfrdBC Δpta) is co-transformed with pIAl and ASKA plasmids.

According to an embodiment of the present invention, the thioesterase is selected from the group consisting of: FadM, YdiI, YbgC, YbfF, YpfH, TesA, TesB, YbaC, YjfP, BioH, YbdB, FrsA, phenylacetyl-CoA thioesterase (PaaI), PaaY, YeiG, and YqiA.

According to an embodiment of the present invention, the itaconate has a yield of 10-40 mg/L within 24 hours.

According to an embodiment of the present invention, the itaconate has a yield up to 150 mg/L in 48 hours using the PaaI.

According to an embodiment of the present invention, the Escherichia coli is cultivated using a microaerobic condition, wherein the microaerobic condition comprises a volume ratio of a cultivation vessel to culture of 6.25:1.

According to an embodiment of the present invention, the PaaI has a specific activity of 0.15-0.19 U/mg towards the itaconyl-CoA.

According to an embodiment of the present invention, the itaconate has a yield up to 1 g/L within 120 hours.

The present invention creates a completely novel itaconate synthesis pathway, which directly uses the central metabolites pyruvate and acetyl-CoA as precursors and can be synthesized through three reactions (see FIG. 1), which has extremely high potential for subsequent optimization and development.

In summary, the present invention provides a novel itaconate synthesis method driven by thioester hydrolysis, which generates a thermodynamic driving force and a kinetic trap by thioesterification reaction, and directs the original reversible pathway to the target itaconate production direction. The present invention replaces the enzyme in the last step of the original pathway (itaconyl-CoA transferase, Ict) with thioesterase, which has never been reported in previous studies.

By identifying the phenylacetyl-CoA thioesterase with itaconyl-CoA hydrolysis activity, compared to the natural fungal route via the TCA cycle, the proposed itaconate pathway exhibits the same maximum theoretical yield from glucose and identical redox balance. However, one additional mole of ATP is generated for every mole of itaconate synthesized from glucose using the reversed degradation pathway, which can be a beneficial trait during microbial biosynthesis. The abundance of pyruvate and acetyl-CoA and decent expression of bacterial enzymes should grant the proposed pathway good potential for development in other unique hosts such as cyanobacteria or methanotrophs.

Currently, the synthetic itaconate pathway appears to be mostly limited by the inefficient hydrolysis of itaconyl-CoA. Engineering thioesterases for better itaconyl-CoA activity while maintaining high specificity will thus be the next essential step to further improve itaconate production using this pathway.

The embodiments of the present invention would be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows schematics of the thioesterase driven production of itaconate using its degradation pathway. The standard change in Gibbs free energy (ΔᵣG′⁰) for each step in the itaconate synthesizing direction was calculated using eQuilibrator and shown in the gray box. Also shown as ΔᵣG′^{m} is the change in Gibbs free energy at substrate concentration of 1 mM calculated by eQuilabrator, which is likely closer to the intracellular state compared to the 1M in the standard case. Abbreviation of each enzyme is shown in the filled circle. Conditions for eQuilibrator calculations were set to pH 7.5 and ionic strength of 0.25M.

FIGS. 2A and 2B show assessment of itaconate performance via its catabolic enzymes. (2A) In vitro verification and comparison of different enzyme candidates found in the itaconate degradation pathway. Purified enzymes were used in various combinations for in vitro itaconate synthesis. (2B) In vivo production of itaconate using YpCcl, PaIch, and YpIct. Strain JCL299 (ΔldhA ΔadhE ΔfrdBC Δpta) was transformed with plasmid pSW41 and allowed to produce for 48 h with or without supplementation of succinate. Error bars represent standard deviation of three duplicates. PA, Pseudomonas aeruginosa; YP, Yersinia pseudotuberculosis; BX, Burkholderia xenovorans; ST, Salmonella enterica serovar typhimurium; RC, Rhodobacter capsulatus; Ccl, citramalyl-CoA lyase; Ich, itaconyl-CoA hydratase; Ict, itaconyl-CoA transferase.

FIGS. 3A and 3B show screening of thioesterase to drive itaconate biosynthesis. (3A) Strain JCL299 (ΔldhA ΔadhE ΔfrdBC Δpta) was co-transformed with plasmids pIAl and pSW32 (harbouring YpIct as negative control), or pIAl and ASKA plasmids containing the corresponding thioesterase genes. Samples were taken after 24 h of cultivation. (3B) Confirmation and comparison of the two best positive thioesterases on itaconate production at 48 h using optimized cultivation condition (glucose feeding and pH adjustment every 12 h). Same transformed strains as FIG. 3A was used. Error bars represent standard deviation of three duplicates.

FIG. 4 shows preliminary assessment of ideal aeration level by varying cultivation vessel and liquid volume. Left panel shows the cultivation set-up with method labels (1) to (7), corresponding to the x-axis on the graph. Strain JCL299 (ΔldhA ΔadhE ΔfrdBC Δpta) transformed with plasmids pIAl and pASKA-paal was used here. Samples were taken after 24 h of incubation. Error bars represent standard deviation of three duplicates.

FIGS. 5A-5C show HPLC preparation and analysis of the CoA thioesters. (5A) Itaconate and acetyl-CoA were incubated with YpIct for 30 min to form itaconyl-CoA. (5B) Following itaconyl-CoA formation by reaction shown in FIG. 5A, PaIch was added to the reaction mixture for 30 min to convert itaconyl-CoA into (S)-citramalyl-CoA. Incubation of acetyl-CoA and itaconate with YpIct and PaIch simultaneously resulted in equally high conversion of acetyl-CoA to (S)-citramalyl-CoA as the sequential reactions shown here and was chosen as the final synthesis method. (5C) Incubation of thioesterase PaaI with itaconyl-CoA prepared from the reaction shown in FIG. 5A.

FIGS. 6A and 6B show improving itaconate production by recycling acetate and minimizing acetate formation. (6A) Different optimization strategies and their resulting itaconate formation at 96 h. Strain JCL299 (ΔldhA ΔadhE ΔfrdBC Δpta) was used as the base strain, of which additional gene knock-outs are indicated by “A” in the table below. Cells transformed with the respective plasmids are labeled with “V” Operon structure is shown next to the name of each plasmid. (6B) Long term itaconate production using high cell density. Time indicates the time since cell concentration and resuspension into fresh medium. Error bars represent standard deviation of three duplicates.

FIG. 7 shows detailed stoichiometry and reaction balance of the two itaconate forming pathways. PEP, phosphoenolpyruvate; OAA, oxaloacetate.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

The present invention is further illustrated by the following examples. These examples are provided for illustration only and are not intended to limit the scope of the present invention. The scope of the present invention is shown in the appended claims.

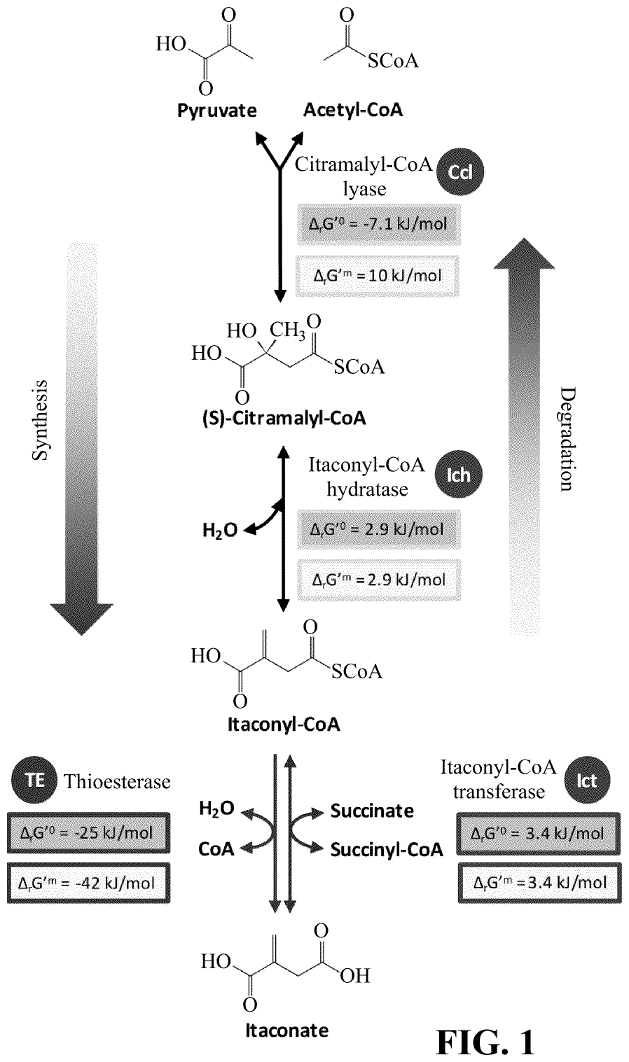

According to the present invention, the present invention demonstrated reversal of the itaconate catabolic pathway for itaconate production by installing an irreversible thioester hydrolysis step in place of the original CoA transfer (FIG. 1). Since no alternative biochemical pathway has been proposed and constructed for itaconate biosynthesis, the present invention took interest in nature's design of itaconate degradation and set out to reverse its reaction sequence via engineering means (FIG. 1).

According to the present invention, inspired by the reversal of β-oxidation pathway via fermentative NADH balance for 1-butanol production, the present invention attempted at creating a synthetic driving force to push carbon flow in the itaconate synthesis direction using the thermodynamically favorable thioester hydrolysis. The use of acyl transfer potential to drive biochemical reactions is commonly found in metabolism. The lower efficiency at resonance stabilization of thioesters results in a greater energy drop from its hydrolysis than the analogous oxygen esters. By substituting the last CoA-transfer step (ΔᵣG′⁰=3.4 KJ/mol) with CoA hydrolysis (ΔᵣG′⁰=−25 KJ/mol) using thioesterase (FIG. 1), the inherently reversible itaconate degradation pathway with an overall ΔG′⁰ of −0.8 KJ/mol was transformed into a thermodynamically favorable pathway with an overall ΔG′⁰ of −29.2 KJ/mol. It is noted that the drop in Gibb's free energy for itaconyl-CoA hydrolysis is even greater when the substrate concentration is set to 1 mM, a level more appropriate physiologically to describe the intracellular metabolites. Acyl-CoA thioesterase belongs to a broad superfamily of thioester hydrolases which catalyze the cleavage of thioester bond of metabolic intermediates found in many essential pathways such as fatty acid and steroid biosynthesis, β-oxidation, and mevalonate metabolism. The wide range of reactions participated thus makes acyl-CoA thioesterase possess a stellar spectrum of substrate range, covering but not limited to saturated/unsaturated fatty acyl-CoA, methyl-branched acyl-CoA, aromatic acyl-CoA, and carboxylic acyl-CoA. The diverse function and structure of thioesterases also make classification difficult; currently the acyl-CoA thioesterases are roughly categorized by their sequence homology, structural domain, and catalytic mechanism, with type I being in the α/β-hydrolase protein family and type II having the signature “hotdog” fold motif. As revealed by many studies, type II acyl-CoA thioesterases typically exhibit broader substrate promiscuity due to the absence of a well-conserved substrate binding pocket, thus allowing them to participate in a wide array of reactions with divergent functions.

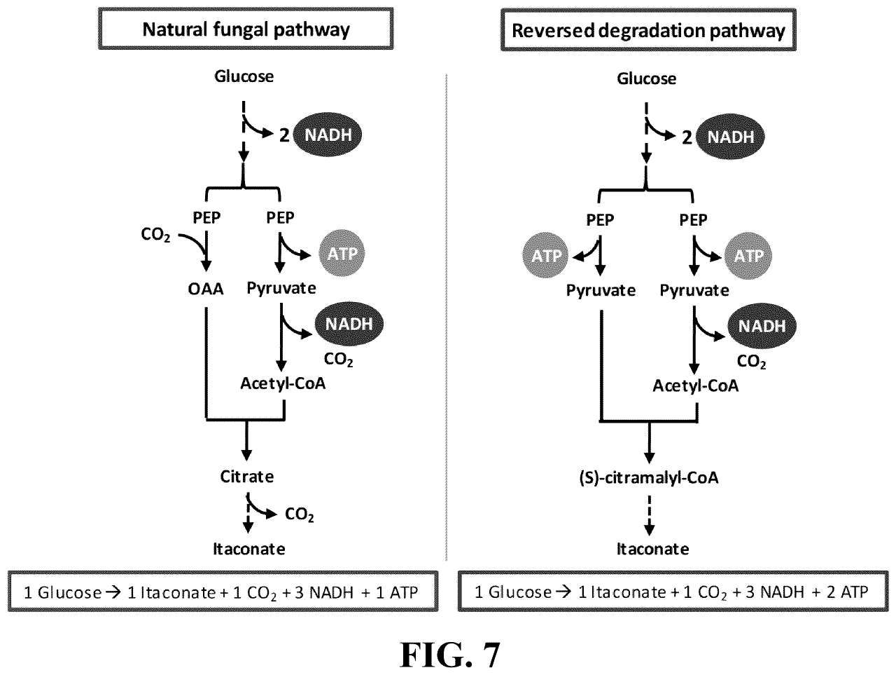

According to the present invention, we engineered the first non-natural itaconate producing pathway driven by thioester hydrolysis. Identification of proper thioesterase with itaconyl-CoA activity was the key factor in reversing the itaconate degradation pathway for biosynthesis. The low preference of target thioesterase towards pathway precursors acetyl-CoA and (S)-citramalyl-CoA was also important to minimize undesired substrate competition and prevent precursor loss. Although the achieved titer and yield are still low compared to the current itaconate production route, our proposed pathway inherently possesses higher energy efficiency that may serve as an attractive trait once fully enhanced. Compared to the natural fungi pathway which generates 1 mole of ATP for every mole of glucose converted to itaconate, the reversed degradation pathway makes 2 mole of ATP due to the distinctive pathway feature (for detailed reaction balance, see FIG. 7). FIG. 7 shows detailed stoichiometry and reaction balance of the two itaconate forming pathways. PEP, phosphoenolpyruvate; OAA, oxaloacetate. The maximum theoretical yield and NADH balance remain the same between the two pathways. While still at its early developmental stage, this study demonstrates a proof-of-concept design of synthetic itaconate pathway based on thermodynamic favorability. The engineering principle and novel itaconate pathway presented here should be readily transferable to other production systems and recombinant hosts.

The reagents and chemicals used in the following examples are as follows. All chemicals and reagents were purchased from Thermo Scientific (Pittsburgh, PA) or Sigma-Aldrich (Saint Louis, MO) unless otherwise specified. Taq DNA ligase, Phusion High-Fidelity DNA polymerase, and T5 exonuclease were obtained from New England Biolabs (Ipswich, MA). Oligonucleotides were purchased from IDT (San Diego, CA). KOD DNA polymerase was purchased from EMD Chemicals (San Diego, CA).

The procedures regarding bacteria strains and DNA manipulation are as follows. Escherichia coli BW25113 was designated as the wild type. XL-1 Blue (Stratagene, La Jolla, CA) was used to propagate all plasmids. Construction of strain JCL16 (BW25113 with lacl^{q} provided on F′) and JCL299 was described in previous study (Atsumi, S., Cann, A. F., Connor, M. R., Shen, C. R., Smith, K. M., Brynildsen, M. P., Chou, K. J., Hanai, T., Liao, J. C., 2008a. Metabolic engineering of Escherichia coli for 1-butanol production. Metabolic engineering. 10, 305-311.) All plasmids were created by the Gibson isothermal DNA assembly method using purified PCR fragments. A list of plasmids used is shown on Table 1. Except for the case of E. coli thioesterases which were obtained from the ASKA (A complete Set of E. coli K12 ORF Archive) plasmid library, genes encoding for the Ict, Ich, Cel, and thioesterase homologues were amplified from the corresponding genomic DNA purchased commercially: Pseudomonas aeruginosa (ATCC 47085D), Yersinia pseudotuberculosis (DSM 8992), Burkholderia xenovorans LB400 (DSM 17367), Salmonella enterica subsp. enterica (DSM17058), Rhodobacter capsulatus (ATCC BAA-309), and Rhodobacter sphaeroides (DSM 5864). PaaI* D16 and H52 saturated variants were created using primers containing the mutated DNA sequence.

TABLE 1

| | |
|---|---|
| Genotype | Reference |

Strain

| | | |
|---|---|---|
| BW25113 | rrnB_{T14} ΔlacZWJ16 hsdR514 ΔaraBAD_{AH33} | Datsenko, K. A., Wanner, |
| | ΔrhaBAD_{LD78} | B. L., 2000. One-step |
| | | inactivation of |
| | | chromosomal genes in |
| | | Escherichia coli K-12 using |
| | | PCR products. Proceedings |
| | | of the National Academy of |
| | | Sciences. 97, 6640-6645. |
| XL-1 Blue | recA1 endA1 gyrA96 thi-1 hsdR17 supE44 relA1 lac | Stratagene |
| | [F′ proAB lacI^{q}ZΔM15 Tn10 (Tet^{R})] | |
| JCL 16 | BW25113/F′ [traD36, proAB⁺, | Atsumi, S., Cann, A. F., |
| | lacI^{q}ZΔM15 (Tet^{r})] | Connor, M. R., Shen, C. R., |
| | | Smith, K. M., Brynildsen, |
| | | M. P., Chou, K. J., Hanai, |
| | | T., Liao, J. C., 2008a. |
| | | Metabolic engineering of |
| | | Escherichia coli for 1- |
| | | butanol production. |
| | | Metabolic engineering. 10, |
| | | 305-311. |
| JCL 299 | JCL 16 ΔldhA ΔadhE ΔfrdBC Δpta | Atsumi, S., Cann, A. F., |
| | | Connor, M. R., Shen, C. R., |
| | | Smith, K. M., Brynildsen, |
| | | M. P., Chou, K. J., Hanai, |
| | | T., Liao, J. C., 2008a. |
| | | Metabolic engineering of |
| | | Escherichia coli for 1- |
| | | butanol production. |
| | | Metabolic engineering. 10, |
| | | 305-311. |
| JCY 298 | JCL299 ΔgltA | Present invention |
| JCY 299 | JCL299 ΔyciA | Present invention |
| JCY 399 | JCL299 ΔyciA ΔgltA | Present invention |

Plasmid

| | | |
|---|---|---|
| pSW26 | P_{T5}::His6X-PaIch; pUC ori; Kan^{R} | Present invention |
| pSW27 | P_{T5}::His6X-Palct; pUC ori; Kan^{R} | Present invention |
| pSW28 | P_{T5}::His6X-PaCcl; pUC ori; Kan^{R} | Present invention |
| pSW29 | P_{T5}::His6X-BxIch; pUC ori; Kan^{R} | Present invention |
| pSW30 | P_{T5}::His6X-BxCcl; pUC ori; Kan^{R} | Present invention |
| pSW31 | P_{T5}::His6X-StIct; pUC ori; Kan^{R} | Present invention |
| pSW32 | P_{T5}::His6X-YpIct; pUC ori; Kan^{R} | Present invention |
| pSW33 | P_{T5}::His6X-YpIch; pUC ori; Kan^{R} | Present invention |
| pSW34 | P_{T5}::His6X-YpCcl; pUC ori; Kan^{R} | Present invention |
| pSW39 | P_{T5}::His6X-RcCcl; pUC ori; Kan^{R} | Present invention |
| pSW41 | P_{L}lacO1::Palch-YpCcl-YpIct; ColE1 ori; Amp^{R} | Present invention |
| pIA1 | P_{L}lacO1::PaIch-YpCcl; p15A ori; Spec^{R} | Present invention |
| pIA16 | P_{T5}::RsMcl2; pUC ori; Kan^{R} | Present invention |
| pIA26 | P_{L}lacO1::Palch-YpCcl-acs; p15A ori; Spec^{R} | Present invention |
| PASKA- | P_{T5-lac}::His6X-TE^{a} ; pBR322 ori; CM^{R} | Present invention |
| TE^{a} | | |
| pASKA- | P_{T5-lac}::His6X-paaI*^{b}; pBR322 ori; CM^{R} | Present invention |
| paaI*^{b} | | |

^{a}TE represents 17 different E. coli genes individually harbored on the ASKA library plasmids (shown as follows): fadM, ydiI, yciA, ybgC, ybfF, ypfH, tesA, tesB, ybaC, yjfP, bioH, ybdB, frsA, paaI, paaY, yeiG, yqiA.
^{b}PaaI* D16 and H52 saturated mutants were cloned onto the same backbone as wild type.
^{c}Abbreviations indicate source of the genes: Pa, Pseudomonas aeruginosa; Bx, Burkholderia xenovorans; St, Salmonella enterica serovar typhimurium; Yp, Yersinia pseudotuberculosis; Rc, Rhodobacter capsulatus; Rs, Rhodobacter sphaeroides.
^{d}Genes with no source abbreviation are derived from Escherichia coli.

The procedures regarding cultivation medium and procedure for itaconate production are as follows. For all of the in vivo itaconate production and screening experiments, single colonies were picked from LB (Luria Broth) plates and inoculated into 2 mL of LB medium contained in test tubes with the appropriate antibiotics (kanamycin 50 μg/mL, spectinomycin 50 μg/mL and chloramphenicol 50 μg/mL). On the next day, the overnight culture grown at 37° C. was inoculated 1% (v/v) into 20 mL of terrific broth (TB) (12 g tryptone, 24 g yeast extract, 2.31 g KH₂PO₄, 12.54 g K₂HPO₄, 4 mL glycerol per liter of water) supplemented with 20 g/L of glucose and appropriate antibiotics in 125 mL screwed-cap flasks (PYREX 4985-125) unless otherwise noted. The culture was allowed to grow at 37° C. in a rotary shaker (250 rpm) to an OD₆₀₀ of 0.4-0.6 then induced with 0.1 mM IPTG and switched to 30° C. Samples were taken and centrifuged to retrieve the supernatant throughout the production period. For itaconate productions using YpIct, 5 g/L of succinate was fed at the time of induction unless otherwise noted.

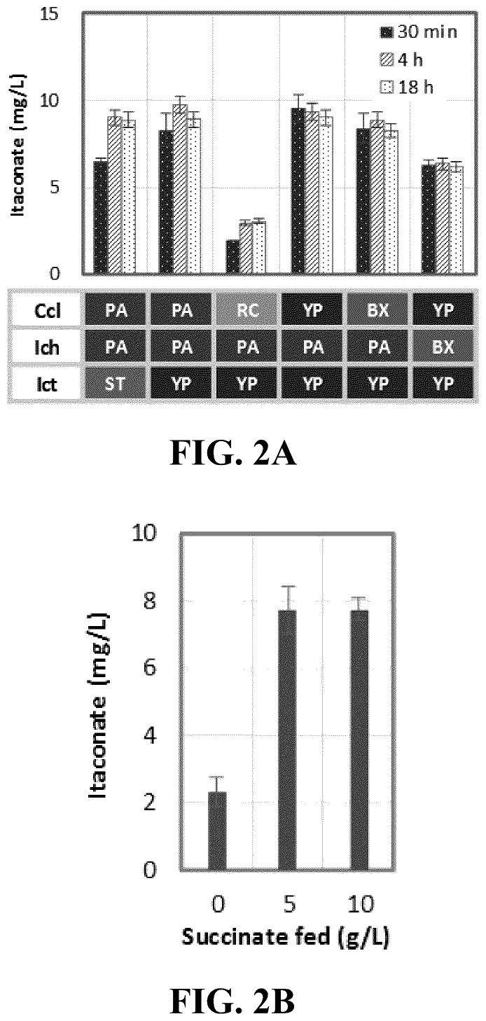

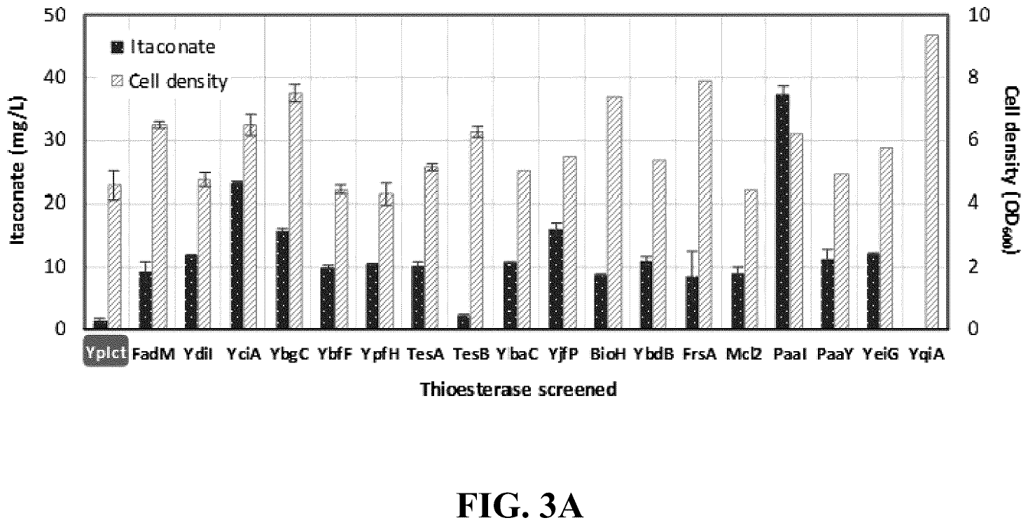

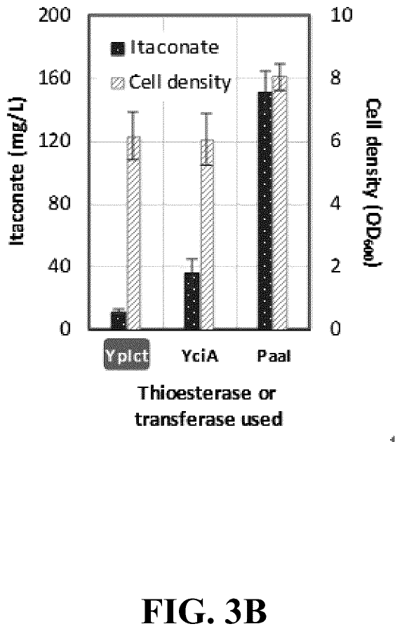

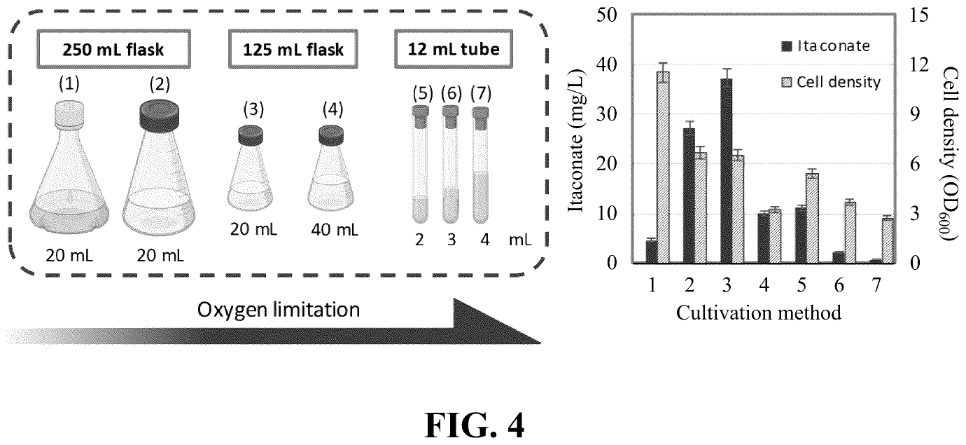

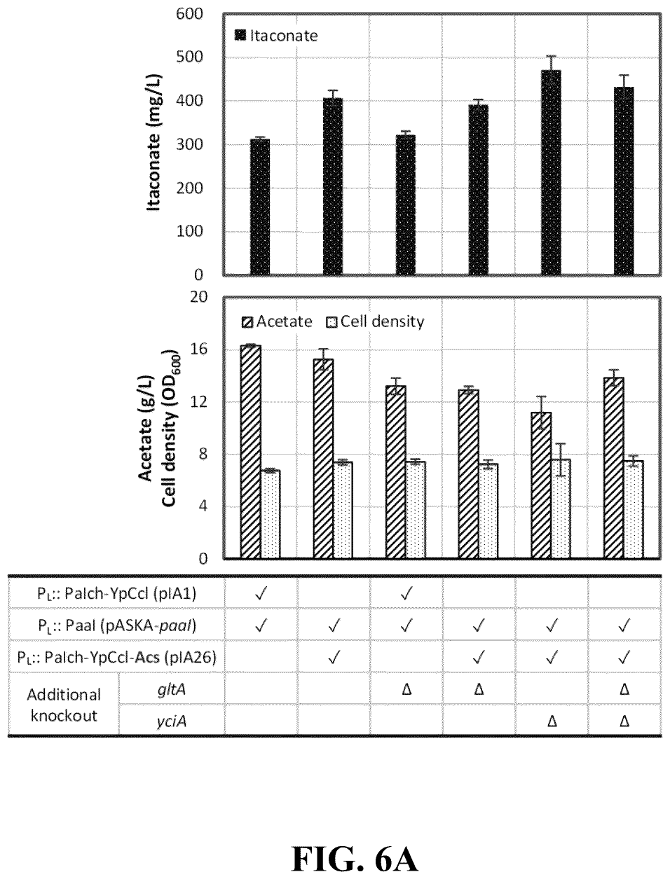

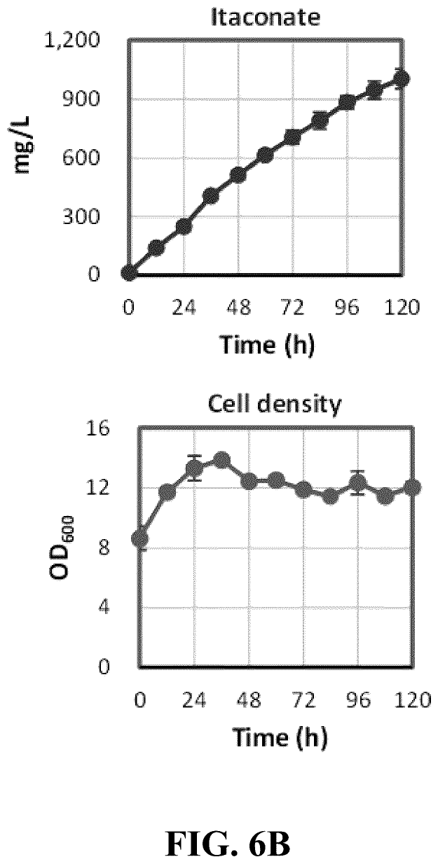

Identical medium and cultivation condition as described above were used in all of the figures except for the modifications outlined below. For FIG. 2B, 250 mL baffled flask (PYREX 4444-250) was used for the production cultivation. For FIG. 4, the inoculated cultures (1% v/v by overnight) were first grown in the 500 mL baffled flasks (PYREX 4444-500) at 37° C. in a rotary shaker (250 rpm) to an OD₆₀₀ of 0.4-0.6. Once reaching exponential phase, the cultures were induced with 0.1 mM IPTG and switched to 30° C. After 4 h of induction at 30° C., the induced cultures were then aliquoted to the different cultivation vessels as indicated on the figure for further itaconate production. The specific vessel product numbers are as follows: vessel 1 is PYREX 250 mL Delong Shaker Erlenmeyer Flask with Baffles (4444-250) with KIMBLE KIM-KAP Polypropylene Cap (73660-38); vessel 2, 3, 4 are PYREX 250/125 mL Narrow Mouth Erlenmeyer Flask with Phenolic Screw Cap (4985-250/125); vessel 5, 6, 7 are BD Vacutainer Serum Tubes (BD 367820). In the case of FIGS. 3B and 6A-6B, glucose was fed to the production culture every 12 h (10 g/L for the first 48 h, then 5 g/L afterwards) with adjustment of culture pH to 7.2 using 10M NaOH or 5M KOH:NH₄OH (1:2, v/v, FIG. 6B). For long-term production on FIG. 6B using concentrated cells, the inoculated cultures were first grown at 37° C. to exponential phase then induced for 4 h at 30° C. using the same vessel and condition as described for FIG. 4. Next, the induced cells were spun down and resuspended with 20 mL fresh TB 2% glucose medium to an initial OD₆₀₀ of about 8, then allowed to produce in 125 mL screwed-cap flasks (PYREX 4985-125) for the next few days at 30° C. and 200 rpm.

The procedures regarding protein purification are as follows. Protein purification was performed using the His-Spin Protein Mini-prep Purification kit from Zymo Research (Irvine, CA). Overnight culture of the XL-1 strains harboring the target enzyme was used to inoculate 20 mL of fresh LB with appropriate antibiotics. The culture was incubated at 37° C. and induced with 0.1 mM IPTG after reaching exponential phase. The induced culture was then switched to 30° C. for protein expression. After 18 to 24 h, the culture was harvested by centrifugation and the resulting pellet was resuspended with 1 mL of His binding buffer suppled in the kit. The resuspended culture was mixed with 1 mL of 0.1 mm glass beads (Biospec) and homogenized using MiniBeadBeater-16, model 607 (Biospec). The soluble fraction was collected by centrifugation at 4° C. The supernatant was run through the His-binding column, washed and eluted with buffer according to the Zymo protocol. The purified protein was mixed with glycerol for storage at −80° C. The protein concentration was determined using Bradford reagent with BSA as standards.

The procedures regarding in vitro itaconate production using purified enzymes are as follows. The reaction mixture contained 100 mM pyruvate, 2.5 mM acetyl-CoA, 100 mM succinate, 5 mM DTT, 5 mM MgCl₂, and 0.5 μM of each purified enzyme in the 0.1 M MOPS-KOH buffer (pH 7.0). Reactions were terminated at various time points by adding 1M HCl. Protein debris was removed by centrifugation then 10M NaOH was added to neutralize the samples to pH 7 for HPLC analysis.

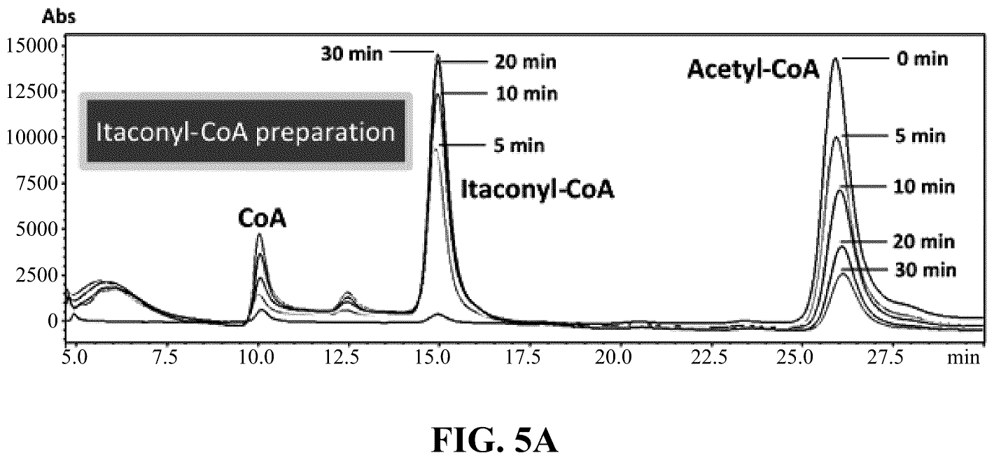

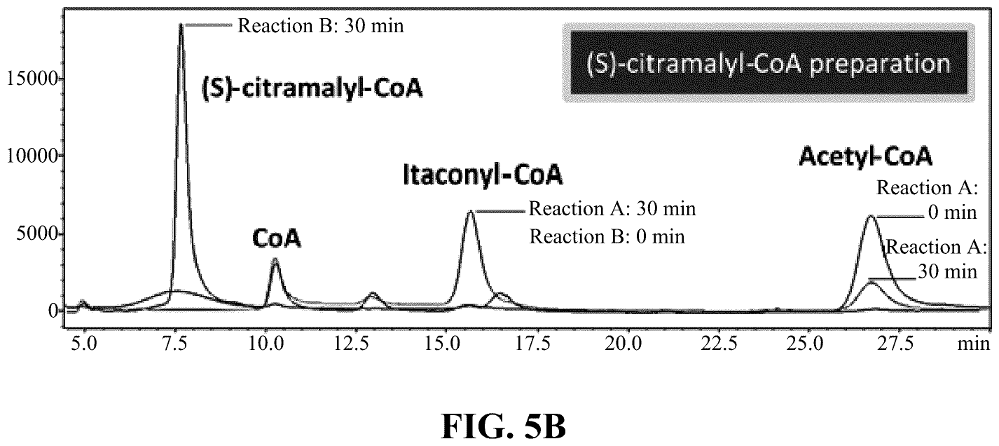

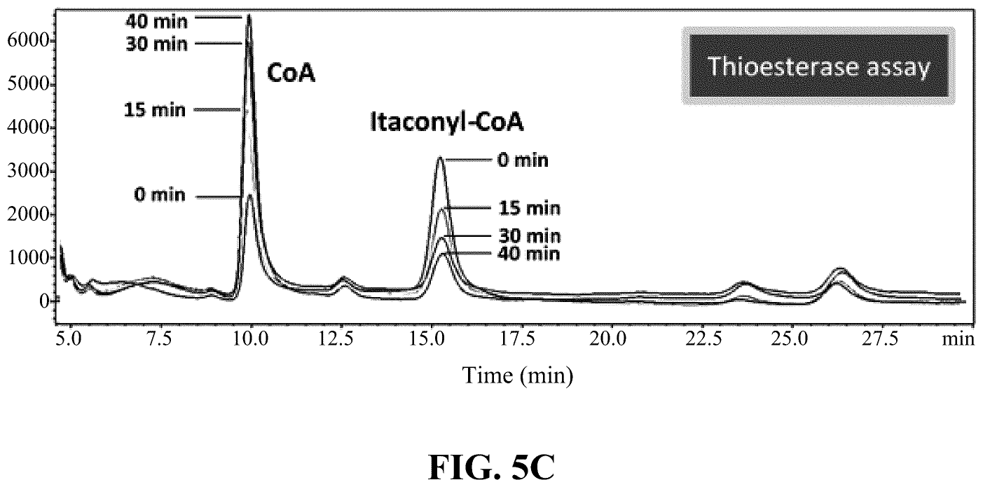

The procedures regarding preparation of CoA intermediates are as follows. Due to the unavailability of commercial itaconyl-CoA and (S)-citramalyl-CoA, enzymatic synthesis of these two CoA intermediates was performed using purified YpIct and PaIch. To synthesize itaconyl-CoA (reaction A), the reaction mixture contained 1 or 2 mM acetyl-CoA, 10 mM itaconate and 0.025 g/L YpIct in 0.1 M MOPS-KOH buffer (pH 7). After incubation at 30° C. for 30 minutes (or various time points as shown on FIGS. 5A-5C), the reaction was terminated by adding 10M HCl to pH around 1. Protein debris was removed by centrifugation at 4° C. followed by pH neutralization to 7 using 10M NaOH. To synthesize(S)-citramalyl-CoA (reaction B), reaction A mentioned above after termination and neutralization was further mixed with 0.025 g/L of PaIch to convert itaconyl-CoA into (S)-citramalyl-CoA. It was later noticed that the preparation of(S)-citramalyl-CoA was equally well achieved by adding YpIct and PaIch simultaneously to reaction A and was chosen as the final preparation method. The reactions were terminated and neutralized using same procedure as mentioned above. The resulting samples were run through Nanosep columns (Pall Life Sciences) then analyzed using HPLC for the different CoA intermediates. Chromatogram peaks corresponding to itaconyl-CoA and (S)-citramalyl-CoA were identified by examining their respective increase and/or decrease in peak area as the target reaction proceeded. Concentration of the synthesized itaconyl-CoA was estimated using the acetyl-CoA absorbance/concentration standard curve and the respective decrease of the acetyl-CoA peak in reaction A. Similarly, concentration of the synthesized(S)-citramalyl-CoA was estimated using the itaconyl-CoA standard curve and the respective decrease of the itaconyl-CoA peak in reaction B. It is noted that certain degree of spontaneous dissociation of CoA from itaconyl-CoA and acetyl-CoA was observed and was subtracted during the calculation of itaconyl-CoA standard curve and kinetic assay of PaaI.

The procedures regarding kinetics assay are as follows. E. coli PaaI activity towards different CoA intermediates was assayed using HPLC by monitoring the decrease of the respective acyl-CoA peak. The reaction A or B mixture obtained from itaconyl-CoA or(S)-citramalyl-CoA preparation was mixed with 0.1 M KCl and purified PaaI (0.02-0.04 g/L). The initial acyl-CoA substrate concentration were as follows: 1 mM acetyl-CoA (commercial), 0.5 mM itaconyl-CoA (estimated from preparation), or 0.8 mM(S)-citramalyl-CoA (estimated by preparation). The reaction was initiated with the addition of PaaI and allowed to proceed at 30° C. At various time points, the reaction was terminated by adding 10M HCl to pH around 1. Protein debris was removed by centrifugation at 4° C. followed by pH neutralization to 7 using 10M NaOH. The resulting samples were analyzed using HPLC.

YpCcl activity towards pyruvate and acetyl-CoA was also assayed using HPLC by monitoring the decrease of the acetyl-CoA peak. The reaction mixture contained 10 mM pyruvate, 1 mM acetyl-CoA and purified YpCcl (0.025 g/L) in 0.1 M MOPS-KOH buffer (pH 7). The reaction was terminated and analyzed using the same procedure as described above.

The procedures regarding quantification of metabolites are as follows. The amount of itaconate, glucose, and other organic acids were quantified using an Agilent 1260 HPLC equipped with an auto-sampler and an Agilent Hi-Plex H column (5 mM H₂SO₄, 0.6 mL/min, column temperature at 50° C.). Glucose was measured with refractive index detector while organic acids were detected using a photodiode array detector at 210 nm. For the identification and analysis of free CoA and CoA esters, Shimadzu Prominence-i LC-2030C 3D Plus HPLC equipped with a GL Sciences C18 column (50 mM KH₂PO₄ at pH 5.3 and methanol (9:1, v/v), 1 mL/min, column temperature at 40° C.) was used. Detection was performed at 259 nm and concentrations were determined by extrapolation from standard curves.

The procedures regarding limiting step assay using YpCcl and PaIch are as follows. The reaction mixture contained 100 mM pyruvate, 2 mM acetyl-CoA, 5 mM DTT, and 5 mM MgCl₂ in 0.1 M MOPS-KOH (pH 7). Purified YpCcl and PaIch were added at 1 or 3 μM as indicated by the ratio “1” or “3”. The reaction was allowed to proceed at 30° C. for 30 min then terminated using the same procedure as described under kinetics assay. Samples were analyzed by HPLC for the resulting itaconyl-CoA and (S)-citramalyl-CoA concentrations.

### Example 1

### Assessment of Itaconate Production Via its Catabolic Enzymes

To examine the inherent possibility of itaconate accumulation via its degradation pathway, we first surveyed the enzyme candidates from the natural itaconate degrading microorganisms. Genes encoding for the citramalyl-CoA lyase (Ccl), itaconyl-CoA hydratase (Ich), and itaconyl-CoA transferase (Ict) from Yersinia pseudotuberculosis (Yp), Pseudomonas aeruginosa (Pa) and Burkholderia xenovorans (Bx) were cloned and overexpressed in E. coli along with a few other homologues identified by protein blast and bioprospecting. Ict from Salmonella enterica serovar typhimurium was selected as an alternative candidate based on its similar pathogenicity and the 76% protein identity with YpIct. Malyl-CoAlyase from Rhodobacter capsulatus (RcCcl), which naturally catalyzes the reversible condensation of glyoxylate and acetyl-CoA or propionyl-CoA, was also tested for its potential unspecific activity towards pyruvate. Based on the thermodynamics calculation of each reaction using the eQuilibrator, the overall Gibbs free energy change of the itaconate catabolic pathway is very close to neutral with no natural push in the synthesis direction (see FIG. 1). Even when all three enzymes are functioning effectively, itaconate formation might be challenging to observe intracellularly without a kinetic trap, especially under strong competition of pyruvate and acetyl-CoA by other essential reactions. Detoxification of itaconate using this pathway was reported to be extremely efficient where high level of itaconate can be degraded with just a catalytic amount of CoA ester. In the case of YpIct which displays relaxed CoA ester specificity, itaconate can be effectively activated by acetyl-CoA, propionyl-CoA, and butyryl-CoA in addition to the natural co-substrate succinyl-CoA. The ability to use acetyl-CoA, the end product of itaconate detoxification, as the CoA donor for next round of itaconate activation circumvents the need of fresh succinyl-CoA supply and further strengthens pathway efficiency in the degradation direction.

To assess the possibility of itaconate production via the degradation enzymes, in vitro biosynthesis of itaconate using purified enzymes was first performed to ensure functional expression of each enzyme and allow saturating concentration of the precursors pyruvate and acetyl-CoA. Different combinations of the bioprospected enzyme homologues were then sequentially checked based on their itaconate formation efficiency.

FIGS. 2A and 2B show assessment of itaconate performance via its catabolic enzymes. (2A) In vitro verification and comparison of different enzyme candidates found in the itaconate degradation pathway. Purified enzymes were used in various combinations for in vitro itaconate synthesis. (2B) In vivo production of itaconate using YpCcl, PaIch, and YpIct. Strain JCL299 (ΔldhA ΔadhE ΔfrdBC Δpta) was transformed with plasmid pSW41 and allowed to produce for 48 h with or without supplementation of succinate. Error bars represent standard deviation of three duplicates. PA, Pseudomonas aeruginosa; YP, Yersinia pseudotuberculosis; BX, Burkholderia xenovorans; ST, Salmonella enterica serovar typhimurium; RC, Rhodobacter capsulatus; Ccl, citramalyl-CoA lyase; Ich, itaconyl-CoA hydratase; Ict, itaconyl-CoA transferase.

As shown on FIG. 2A, the highest accumulation of itaconate in vitro was achieved by YpCcl, PaIch, and YpIct, reaching nearly 10 mg/L in 30 min. Other homologue combinations led to 10-80% lower level of itaconate formation with RcCcl performing the worst, suggesting its low substrate preference towards pyruvate despite the high structural similarity with its natural substrate glyoxylate. Next, to further examine the potential of accumulating itaconate based on the intracellular concentration of pyruvate and acetyl-CoA, we cloned the best performing enzymes into an operon (PaIch-YpCcl-YpIct, plasmid pSW41) and attempted itaconate synthesis in vivo by overexpressing this catabolic pathway in the fermentation deleted strain JCL299 (ΔldhA ΔadhE ΔfrdBC Δpta). Elimination of fermentative reactions was consistently shown to improve biochemical productions derived from pyruvate and/or acetyl-CoA such as isobutanol, 1-butanol, and 2,3-butanediol. In this example, use of JCL299 allowed approximately four-fold higher level of itaconate formation (˜8 mg/L) compared to the titer achieved using wild type E. coli. As one can see from FIGS. 2A and 2B, similar level of itaconate production was observed in vitro and in vivo with addition of succinate as the essential co-substrate for the itaconyl-CoA transferase. This observation indicates the sufficiency of intracellular pyruvate and acetyl-CoA concentration to direct carbon flux into itaconate via the highly reversible pathway. The low accumulation of itaconate, however, in both the in vivo and in vitro cases appeared to have originated from the pathway reversibility and a lack of driving force. Design of a synthetic push to further drive carbon flux into the target itaconate is thus pursued next.

### Example 2

### Screening of Thioesterases to Drive Itaconate Production

In previous studies, accumulation of NADH and pathway rewiring with ATP dependent reactions have successfully channeled carbon flux towards 1-butanol production via the highly reversible β-oxidation pathway. However, the itaconate degradation pathway does not exhibit any cofactor or redox dependency nor nutrition auxotrophic characteristics that can be directly implemented as driving force. Since metabolic pathways found in nature such as fatty acid biosynthesis are often driven by the acyl transfer potential, substitution of CoA transferase with thioesterase should provide an effective kinetic trap to irreversibly push carbon into itaconate. Since no thioesterase has been specifically characterized for itaconyl-CoA activity, here we bioprospected for endogenous thioesterases in E. coli for their ease of expression and potential substrate promiscuity as elimination of native thioesterase is often necessary to minimize hydrolysis of CoA intermediates into acid byproducts. In addition to the ones involved in fatty acid biosynthesis, E. coli possesses a wide range of thioesterases with unclear physiological function. A total of 17 enzymes (FadM, YdiI, YciA, YbgC, YbfF, YpfH, TesA, TesB, YbaC, YjfP, BioH, YbdB, FrsA, PaaI, PaaY, YeiG, YqiA) annotated or reported with thioesterase activity was selected along with (3S)-malyl-CoA thioesterase (Mcl2) from Rhodobacter sphaeroides for its potential activity towards itaconyl-CoA. Strain JCL299 harboring plasmids pIAl (PLlac01::PaIch-YpCcl) and the different thioesterases overexpressed from pBR302 backbone was then cultivated under microaerobic condition, and the resulting itaconate formation was compared.

FIGS. 3A and 3B show screening of thioesterase to drive itaconate biosynthesis. (3A) Strain JCL299 (ΔldhA ΔadhE ΔfrdBC Δpta) was co-transformed with plasmids pIAl and pSW32 (harbouring YpIct as negative control), or pIAl and ASKA plasmids containing the corresponding thioesterase genes. Samples were taken after 24 h of cultivation. (3B) Confirmation and comparison of the two best positive thioesterases on itaconate production at 48 h using optimized cultivation condition (glucose feeding and pH adjustment every 12 h). Same transformed strains as FIG. 3A was used. Error bars represent standard deviation of three duplicates.

As shown on FIG. 3A, nearly all thioesterase candidates demonstrated certain degree of hydrolytic activity towards itaconyl-CoA as reflected by the 10 to 40 mg/L of itaconate secretion in 24 h. YpIct overexpressed from a similar backbone system as the thioesterases was used here as a negative control for comparison.

The thioesterases which delivered higher level of itaconate (>15 mg/L) were identified to be YciA, YbgC, YjfP and PaaI. It is interesting to note that three of the enzymes except YjfP belong to the type II thioesterase class which was demonstrated to exhibit broad substrate activity. Of the two highest itaconate producing thioesterases, YciA was reported to possess a wide spectrum of substrate promiscuity ranging from short-chain (such as acetyl-CoA, isobutyryl-CoA) and long chain (such as n-decanoyl-CoA, oleoyl-CoA) aliphatic acyl-CoA to phenyl acyl-CoA substrates. On the other hand, PaaI found in the phenylacetate degradation pathway is generally considered to hydrolyze phenylacetyl-CoA and its mono- and di-hydroxy ring analogues, rather than aliphatic acyl-CoA thioesters. To further confirm the superior performance of PaaI, itaconate productions using thioesterase PaaI and YciA were repeated under optimized cultivation condition and pH adjustment, with the CoA transferase YpIct as control comparison (FIG. 3B). It is noted that the redox neutral property of the reversed itaconate degradation pathway puts oxygen demand at a challenging position. Lower supply of oxygen is typically ideal to maintain higher pool of acetyl-CoA and pyruvate in the fermentation deleted strain (JCL299). However, the need to recycle reducing power in JCL299 makes respiration essential.

FIG. 4 shows preliminary assessment of ideal aeration level by varying cultivation vessel and liquid volume. Left panel shows the cultivation set-up with method labels (1) to (7), corresponding to the x-axis on the graph. Strain JCL299 (ΔldhA ΔadhE ΔfrdBC Δpta) transformed with plasmids pIA1 and pASKA-paal was used here. Samples were taken after 24 h of incubation. Error bars represent standard deviation of three duplicates.

As shown on FIG. 4, preliminary assessment of ideal aeration level by varying cultivation vessel and liquid volume indicated that microaerobicity (20 mL culture in 125 mL non-baffled screw cap flask) enabled the best itaconate production. On the other hand, oxygen supply on the extreme ends (fully aerated in baffled flask and minimal aeration in sealed tube with high culture volume) both resulted in significantly lower accumulation of itaconate. At last, implementation of the optimal aeration condition and pH adjustment greatly elevated itaconate formation to 150 mg/L in 48 h using PaaI (FIG. 3B), which was about four and 15 fold higher than the amount achieved by YciA and the transferase YpIct respectively.

### Example 3

### Kinetic Analysis of Thioesterase PaaI for the CoA Intermediates in the Itaconate Pathway

As reported in prior studies, many type II thioesterases possess unspecific activity toward a wide range of acyl-CoA substrates. Since the itaconate degradation pathway involves three different acyl-CoA intermediates, namely acetyl-CoA, (S)-citramalyl-CoA, and itaconyl-CoA, we characterize the thioesterase activity of our positive candidate PaaI towards each of the CoA substrates. Due to the unavailability of(S)-citramalyl-CoA and itaconyl-CoA commercially, we first pursued the in vitro preparation of these two CoA compounds by the itaconate degradation reactions.

FIGS. 5A-5C show HPLC preparation and analysis of the CoA thioesters. (5A) Itaconate and acetyl-CoA were incubated with YpIct for 30 min to form itaconyl-CoA. (5B) Following itaconyl-CoA formation by reaction shown in FIG. 5A, PaIch was added to the reaction mixture for 30 min to convert itaconyl-CoA into (S)-citramalyl-CoA. Incubation of acetyl-CoA and itaconate with YpIct and PaIch simultaneously resulted in equally high conversion of acetyl-CoA to (S)-citramalyl-CoA as the sequential reactions shown here and was chosen as the final synthesis method. (5C) Incubation of thioesterase PaaI with itaconyl-CoA prepared from the reaction shown in FIG. 5A.

As shown by the HPLC chromatograms (FIGS. 5A-5C), incubation of itaconate and acetyl-CoA with the CoA transferase YpIct or in combination with the hydratase PaIch resulted in the formation of distinct new peaks using the CoA retention capable C18 column. Acetyl-CoA was used here as the CoA donor for its comparable efficiency as succinyl-CoA partnering with YpIct. When only YpIct was present, the new peak around 15.5 min gradually enlarged while the acetyl-CoA peak shrunk with similar aspect ratio as time progressed from 0 to 30 min. Subsequent addition of PaIch after observation of the 15.5 min peak led to appearance of the 7.6 min peak with drop of the 15.5 min peak area. Based on the respective reaction sequence and peak behavior, we then identified the 15.5 min peak as itaconyl-CoA and the 7.6 min peak as (S)-citramalyl-CoA. The (S)-citramalyl-CoA peak was further confirmed by incubation of the citramalyl-CoA lyase YpCcl with pyruvate and acetyl-CoA, which resulted in the gradual appearance of the 7.6 min peak with consumption of acetyl-CoA.

With successful preparation and identification of the CoA intermediates, we proceeded with kinetic assay of the thioesterase PaaI. Contrary to the general observation of broad substrate range associated with type II thioesterases, PaaI displayed relatively low activity towards itaconyl-CoA (0.17 U/mg, U: μmol/min) and even lower activity towards acetyl-CoA (0.015 U/mg) compared to the level reported for its most active hydroxyphenylacetyl-CoA substrates. In addition, no activity was detected towards the pathway intermediate (S)-citramalyl-CoA, which was also reflected by no accumulation of citramalate in the itaconate production broth. Based on our kinetics analysis, the (S)-citramalyl-CoA lyase YpCcl demonstrated suboptimal but higher activity towards acetyl-CoA compared to the level observed in PaaI (Table 2).

TABLE 2

| Enzyme | Substrate | Specific activity (U/mg) |
|---|---|---|
| PaaI | Acetyl-CoA | 0.015 ± 0.006 |
| | (S)-Citramalyl-CoA | Not detected (ND) |
| | Itaconyl-CoA | 0.17 ± 0.02 |
| YpCcl | Acetyl-CoA + Pyruvate | 0.06 ± 0.02 |

This skewed preference of PaaI towards itaconyl-CoA over the other CoA intermediates in the pathway helped minimize loss of key precursors to unspecific hydrolysis and contributed significantly to the superior performance of PaaI in itaconate production. E. coli PaaI is hypothesized to have evolved from the same superfamily with the Arthrobacter 4-hydroxyphenacyl-CoA thioesterase. Structural and activity analysis revealed that it is functionally unrelated to the benzoate-based metabolic pathways and exhibits undetectable to minimal activity towards benzoyl-CoA and aliphatic acyl-CoA thioesters respectively. It is noted that extremely low activity towards C2 to C6 saturated aliphatic thioesters such as acetyl-CoA, butyryl-CoA, and hexanoyl-CoA was also observed from the Arthrobacter PaaI homologue, along with methylmalonyl-CoA which structurally resembles itaconyl-CoA in terms of its carboxylated and methylated feature. Our finding of the relatively decent itaconyl-CoA activity from the E. coli PaaI thioesterase adds to the insight of its substrate spectrum. Itaconate production via this CoA-bound pathway should benefit greatly from the specificity of PaaI given that itaconyl-CoA activity can be elevated without lifting the limited activity towards the other CoA intermediates.

### Example 4

### Acetate Recycle and Minimization

During the optimization of itaconate production, significant secretion of acetate was observed (>10 g/L), suggesting insufficiency of the reversed degradation pathway to sequester the high flux of pyruvate and acetyl-CoA. Small amount of pyruvate was also noted in the production media (˜1 g/L). As mentioned previously, microaerobic cultivation was used here for itaconate biosynthesis to allow accumulation of precursors and NADH oxidation via respiration in the fermentation deleted strain JCL299. Examination of the (S)-citramalyl-CoA lyase (YpCcl) catalyzing the reversible condensation of pyruvate and acetyl-CoA revealed its relatively low activity (0.06 U/mg, Table 2) compared to other endogenous competing reactions. The suboptimal reaction rate thus might have contributed to the overflow of acetate. Limited supply of oxygen might further push the ATP-producing acetate formation which cannot be eradicated with just pta deletion.

Initially, elimination of citrate synthase (gltA) responsible for the entrance of TCA cycle was performed to conserve acetyl-CoA in the attempt to boost the condensation rate by YpCcl. However, itaconate level stayed the same. Since acetate excretion is commonly observed in E. coli fermentation due to metabolic overflow, the most viable approach is to recycle the acetate back to acetyl-CoA. This should also help convert back any acetyl-CoA that was lost to acetate due to unspecific thioesterase activity present in the cell. Indeed, overexpression of the acetyl-CoA synthetase (Acs) led to a 30% increase in itaconate production; however, similar secretion level of acetate was observed. Further survey of literature suggested that the highly-efficient promiscuous activity of the native thioesterase YciA towards acetyl-CoA often contributes to the accumulation of acetate.

FIGS. 6A and 6B show improving itaconate production by recycling acetate and minimizing acetate formation. (6A) Different optimization strategies and their resulting itaconate formation at 96 h. Strain JCL299 (ΔldhA ΔadhE ΔfrdBC Δpta) was used as the base strain, of which additional gene knock-outs are indicated by “A” in the table below. Cells transformed with the respective plasmids are labeled with “V” Operon structure is shown next to the name of each plasmid. (6B) Long term itaconate production using high cell density. Time indicates the time since cell concentration and resuspension into fresh medium. Error bars represent standard deviation of three duplicates.

As shown by FIG. 6A, deletion of the endogenous YciA not only reduced acetate formation but also further enhanced itaconate production by 15% compared to the level achieved in strain JCL299. It is interesting to note that YciA was the second best candidate that led to better itaconate formation compared to the other endogenous thioesterases screened. The high activity of YciA towards acetyl-CoA can be a big disadvantage for the synthesis of itaconate using this CoA-bound pathway. It is thus essential to keep in mind that low preference for acetyl-CoA and the other CoA intermediates must be maintained while engineering thioesterases for better itaconyl-CoA activity.

With the preliminary metabolic pathway tuning finished, we proceeded with condition optimization. From a series of aeration, pH, intermittent glucose feeding, and cell density testing (for detailed condition, see Methods), we achieved an itaconate titer of 1 g/L in 120 h (as shown in FIG. 6B), which is nearly 100-fold increase compared to the initial level obtained using the CoA transferase. Even though current production rate is still suboptimal, here we successfully demonstrated the effectiveness of installing a thermodynamic trap for setting the pathway direction. Relative to the irreversible thioester hydrolysis in the last step, the first two reactions may now become the limitation for the itaconate formation rate.

Incorporation of irreversible steps in pathway design enhances the overall thermodynamic favorability and often leads to better bioconversion yield given functional enzymes. Using this concept, the present invention constructed the first non-natural itaconate biosynthesis pathway driven by thioester hydrolysis. Itaconate is a commercially valuable platform chemical with wide applications in the synthetic polymer industry. Production of itaconate has long relied on the decarboxylation of TCA cycle intermediate cis-aconitate as the only biosynthetic route. Inspired by nature's design of itaconate detoxification, here we engineered a novel itaconate producing pathway orthogonal to native metabolism with no requirement of auxotrophic knock-out.

FIG. 7 shows detailed stoichiometry and reaction balance of the two itaconate forming pathways. PEP, phosphoenolpyruvate; OAA, oxaloacetate. The maximum theoretical yield and NADH balance remain the same between the two pathways. The reversed degradation pathway initiates with pyruvate and acetyl-CoA condensation forming(S)-citramalyl-CoA, followed by its dehydration and isomerization into itaconyl-CoA then hydrolysis into itaconate. Phenylacetyl-CoA thioesterase (PaaI) from Escherichia coli was identified via screening to deliver the highest itaconate formation efficiency when coupled to the reversible activity of citramalate lyase and itaconyl-CoA hydratase. The preference of PaaI towards itaconyl-CoA hydrolysis over acetyl-CoA and (S)-citramalyl-CoA also minimized the inevitable precursor loss due to enzyme promiscuity. With acetate recycling, acetyl-CoA conservation, and condition optimization, we achieved a final itaconate titer of 1 g/L using the thioesterase driven pathway, which is a significant improvement compared to the original degradation pathway based on CoA transferase. The present invention illustrates the significance of thermodynamic favorability as a design principle in pathway engineering.

In summary, the present invention provides a novel itaconate synthesis method driven by thioester hydrolysis, which has yet been reported in prior studies. By identifying the phenylacetyl-CoA thioesterase with itaconyl-CoA hydrolysis activity, we successfully rewired the itaconate detoxification pathway for itaconate biosynthesis. Compared to the natural fungal route via the TCA cycle, the proposed itaconate pathway exhibits the same maximum theoretical yield from glucose and identical redox balance. However, one additional mole of ATP is generated for every mole of itaconate synthesized from glucose using the reversed degradation pathway, which can be a beneficial trait during microbial biosynthesis. Although the present itaconate titer and yield are far below the traditional production level, the abundance of pyruvate and acetyl-CoA and decent expression of bacterial enzymes should grant the proposed pathway good potential for development in other unique hosts such as cyanobacteria or methanotrophs. Currently, the synthetic itaconate pathway appears to be mostly limited by the inefficient hydrolysis of itaconyl-CoA. Engineering thioesterases for better itaconyl-CoA activity while maintaining high specificity will thus be the next essential step to further improve itaconate production using this pathway.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for synthesizing itaconate driven by thioesterase, comprising initiating pyruvate and acetyl-CoA condensation to form (S)-citramalyl-CoA through a reversed degradation pathway, followed by dehydration and isomerization of the (S)-citramalyl-CoA into itaconyl-CoA, and then hydrolysis of the itaconyl-CoA into itaconate driven by thioesterase.

2. The method according to claim 1, wherein the thioesterase is from Escherichia coli.

3. The method according to claim 2, wherein the Escherichia coli is Escherichia coli strain JCL299 (ΔldhA ΔadhE ΔfrdBC Δpta).

4. The method according to claim 3, wherein the Escherichia coli strain JCL299 (ΔldhA ΔadhE ΔfrdBC Δpta) is co-transformed with pIAl and ASKA plasmids.

5. The method according to claim 1, wherein the thioesterase is selected from the group consisting of: FadM, YdiI, YbgC, YbfF, YpfH, TesA, TesB, YbaC, YjfP, BioH, YbdB, FrsA, phenylacetyl-CoA thioesterase (PaaI), PaaY, YeiG, and YqiA.

6. The method according to claim 1, wherein the itaconate has a yield of 10-40 mg/L within 24 hours.

7. The method according to claim 5, wherein the itaconate has a yield up to 150 mg/L in 48 hours using the PaaI.

8. The method according to claim 2, wherein the Escherichia coli is cultivated using a microaerobic condition, wherein the microaerobic condition comprises a volume ratio of a cultivation vessel to culture of 6.25:1.

9. The method according to claim 5, wherein the PaaI has a specific activity of 0.15-0.19 U/mg towards the itaconyl-CoA.

10. The method according to claim 1, wherein the itaconate has a yield up to 1 g/L within 120 hours.
